(19) United States
(12) Patent Application Publication
(10) Pub. No.: US 2011/0112380 A1
(43) Pub. Date: May 12, 2011

(54) Method and System for Optimal Estimation in Medical Diagnosis

(75) Inventor: John M. Robinson, Brookings, SD (US)

(73) Assignee: eTenum, LLC, Brookings, SD (US)

(21) Appl. No.: 12/945,670

(22) Filed: Nov. 12, 2010

Related U.S. Application Data
(60) Provisional application No. 61/260,641, filed on Nov. 12, 2009.

(51) Int. Cl. A61B 5/00

(52) U.S. Cl. 600/300

(57) ABSTRACT

A system and method for estimating and updating the current state of disease of a patient, called the disease impression. The disease impression of the patient, for a disease with a set of possible disease conditions, is updated based upon the current disease impression, the outcome of a test performed with respect to the disease in the patient, and the conditional probabilities of obtaining the test outcome given each of the disease conditions for the disease. The conditional probabilities with respect to all possible outcomes of the test and conditions of the disease may be stored in a likelihood matrix, which comprises a medical knowledge base. Because tests are error prone, the estimate of the patient's state of disease evolves according to a hidden Markov model, which allows the clinical impression of the patient to be computed in real-time. By applying the method to many diseases and many tests, large-scale medical diagnosis is achieved and is computationally tractable because as a result of the process, such diagnosis becomes stochastic filtering problem that is trivially parallel. In addition, the expected diagnostic utility of any test can be predicted. The same knowledge base is used to perform both inference and prediction.

### PRIORITY CLAIM

This application claims priority to and the benefit of the provisional patent application entitled Optimal Estimation in Medical Diagnosis, application Ser. No. 61/260,641, filed Nov. 12, 2009.

### FIELD OF INVENTION

The present invention relates to the field of medical diagnosis, and in particular to systems and methods for estimating the state of disease(s) in a patient based upon the outcomes of tests that are subject to error. The method may be scaled in parallel across a plurality of tests and a plurality of diseases to achieve the clinical impression of the patient.

### BACKGROUND

Medical diagnosis is the process of translating clinical findings into judgments about the health of a person. Medical diagnosis is complex because the quality and relevance of clinical data must inform an evolving impression of a person's health within an adaptive strategy for generating new data. Medical diagnosis requires reasoning based on incomplete, uncertain, and changing information since medical testing is constrained by concerns about time, cost, and safety; since tests can produce erroneous outcomes; and since the patient condition and patient data can evolve. These, along with the explosion of information that is available from advances in biomedical research, laboratory medicine, imaging studies, and high-throughput “omic” methods—genomic, proteonomic, metabolomic—challenge our capacity to distill clinical information into an understanding of the patient, especially one that is transparent to clinicians, nurses, the patient, care givers, insurers, and others. This challenge is a barrier to two imperatives in modern medicine: applying population-based evidence to patient-specific diagnosis (so called evidence-based medicine) and implementing a comprehensive electronic medical record (EMR) system that treats the value of the information about a person.

Since the 1950's, investigators have sought a paradigm where the computer can function as a quantitative tool to support the cognitive processes of clinicians who are engaged in large-scale medical diagnosis—the simultaneous diagnosis of multiple diseases among the complete inventory of medical diseases. The paradigm should support an approach to medical diagnosis with 4 characteristics: Medical diagnosis is a dynamic real-time process; the clinician's disease impression of disease is probabilistic rather than deterministic; the disease impression is updated given new clinical findings; and medical tests are subject to error. Despite over five decades of research, investigators have been unable to define a paradigm that is both computationally tractable and implements the four characteristics of medical diagnosis. Early models of medical diagnosis supported deterministic reasoning. These included hypothetico-deductive models, decision trees, and heuristic pattern recognition models. Deterministic models were adapted to support probabilistic reasoning: Decision trees were extended using fuzzy set theory; heuristic models were extended using confirmation theory and recast as Bayesian networks. Among these, Bayesian networks seemed most promising, but solving for the probabilities of interest is not feasible for large systems, whether the solution is exact or approximate. Modified (or reduced) networks of the parent Bayesian network were sought. These were attempts to discover alternative probability-based models that treated the temporal, iterative, and adaptive nature of diagnosis and were also computationally tractable. Previous research has not addressed the forth characteristic of medical diagnosis—that medical tests are subject of error. Thus, there exists a need for a computationally tractable method that implement the four characteristics of medical diagnosis and which may be practically implemented in computer software and systems.

### SUMMARY OF THE INVENTION

Developments in ontologies as building blocks for semantic webs offer new approaches for scientific analysis and for translational research in biomedicine. Here, embodiments of the present invention provide a system and method for medical diagnosis that implement the four characteristics of medical diagnosis, including the problem of test error and its effect on the reliability of diagnoses. The logic of large-scale medical diagnosis is shown to be a Diagnostic Semantic Web (DSW) that is assembled from the semantic triples (ontologies) of different disease/test combinations. The DSW is a quantitative model that may be implemented in software and distributed across computer networks and systems to clinicians engaged in medical diagnosis. A clinician's estimate of the state of a disease in a person (called the disease impression) is a probability distribution that evolves in response to test outcomes. When tests are conditionally independent, the DSW is equivalent to a set of hidden Markov models, where each hidden Markov model governs the evolution of its disease-specific disease impression. In this way, embodiments of the present invention treat medical diagnosis as a parallel stochastic filtering problem. Medical diagnosis becomes computationally tractable since it is trivially parallelizable. When tests are not conditionally independent, the approach provides satisfactory results but is computationally more complex.

The principles of the present invention result in quantitative methods for two essential tasks in medical diagnosis: inference and prediction. The inference method quantifies in real time the diagnostic value of test outcomes as they arrive. The prediction method provides the expected diagnostic benefit of a putative test given the current state of the diagnostic evaluation. As a layer of logic within a next generation EMR system, these methods support an easily understandable and self-documenting account of the strategy and reasoning that led to the current view of a patient's state of health, including diagnoses and confidence in the diagnoses. The clinical impression—the set of disease impressions for all diseases in the medical lexicon—provides a unified, quantitative, and transferable best estimate of a patient's state of health.

One embodiment of a method of the present invention comprises inferring the impression of the state of a disease in a patient, for a disease with a set of possible disease conditions, comprising identifying a current disease impression of the patient; obtaining the outcome of a test performed with respect to the disease in the patient; and updating the current disease impression to a new disease impression based upon the conditional probabilities of obtaining the test outcome given each of said disease conditions. A test has a set of possible outcomes, and therefore a likelihood matrix can be comprising the conditional probability distribution of obtaining each test outcome given each of the disease conditions. These conditional probabilities are used in updating the disease impression. The method can be extended to a plurality of tests for a plurality of diseases, in which case a likelihood matrix is provided with the conditional probability distribution of each test outcome given each disease condition for each disease. The disease impression preferably updated from test results until it is within a predetermined threshold of confidence with respect to one of the disease conditions for the disease of interest. If repeated with respect to a plurality of diseases, the clinical impression of the patient is obtained.

One embodiment of a system of the present invention comprises a database stored in a computer readable memory comprising a plurality of likelihood matrices for a plurality of diseases and a plurality of tests, each such disease comprising a set of disease conditions and each such test comprising a set of possible test outcomes, so that each likelihood matrix comprises, for one of the diseases and one of the tests, the conditional probability distribution of each test outcome given each disease condition. The system also includes a processor in communication with the database, the processor having a computer readable memory storing instructions executable by said processor that identify a current disease impression of a patient; obtain the outcome of a test performed with respect to the disease in the patient; retrieve from the database the conditional probabilities of obtaining the test outcome given each of the disease conditions; and update the current disease impression to a new disease impression based upon the conditional probabilities. The database may contain likelihood matrices for a plurality of diseases and a plurality of tests, in which case the system can update the clinical impression of the patient in response to one or more test outcomes.

### DESCRIPTION OF THE FIGURES

The present invention will be explained, by way of example only, with reference to certain embodiments and the attached Figures, in which:

FIG. 1 is a diagram of a cognitive model of medical diagnosis.

FIG. 2 is an illustration of an exemplary diagnostic space for a disease;

FIG. 3(a) is a diagram of the process of medical diagnosis as a diagnostic web, which is a bipartite semantic web.

FIG. 3(b) illustrates an embodiment of one aspect of a method of the present invention, showing that each disease impression pⱼ in the diagnostic web updates as a hidden Markov model;

FIG. 4 is an example of a diagnosis of a binary disease by repeated application of a (2×2)-test.

FIG. 5 is an exemplary comparison of two (2×2)-tests to illustrate that test utility depends on the current disease impression.

FIG. 6 illustrates an example of paradoxical slowing down of diagnosis, with identical conditions as in FIG. 5 except the initial disease impression favors non-infection.

FIG. 7 is a diagram of one embodiment of a method of the present invention;

FIG. 8 is a diagram of one embodiment of a system of the present invention; and

FIG. 9 is a diagram of an alternative view one embodiment of a system of the present invention.

### DETAILED DISCUSSION

### I. Definitions

Allopathic medicine flows from a fundamental axiom: A disease causes (a set of) observable clinical features,

disease→{features}. (1)

This statement roots medicine in empirical science. The task of biomedical research is to characterize the details of the statement. An important task in electronic medical record systems is to translate natural language expressions of diseases and observable clinical features into expressions that involve standardized medical terminology such as ICD-9, ICD-10, SNOMED-CT. The task of medical diagnosis is to evaluate Eq. 1 in the reverse direction—to infer the state of disease in a person from tests that measure the features of a disease. When causality is not absolute but suggestive, Eq. 1 is understood as a probabilistic expression. Methods in probabilistic inference invariably make use of Bayes theorem, but the methods differ in how Bayes theorem is applied.

### A. Disease

Large-scale medical diagnosis is concerned with the simultaneous diagnosis of multiple diseases. To be able to quantitatively support medical diagnosis, we must first define elementary data types. Here, the state of a particular disease in a person is described by the state variable X. The state variable X is in one of n≧2, preferably mutually exclusive and exhaustive, disease conditions Ω_{X}={x₀, . . . , xₙ₋₁}={xᵢ}. The disease conditions are dictated by current standards in medical practice. They are ordered in ascending severity beginning with the null disease condition x₀. Examples:

1. Infection: not infected (x₀), infected (x₁).

2. Hypertension: normotensive (x₀), mild (x₁), moderate (x₂), severe (x₃).

Example 1 illustrates the classical yes-no (binary) description of disease, where a person either suffers from the disease (x₁) or does not suffer from the disease (x₀). Including the null disease condition x₀ in the set of disease conditions Ωₓ supports strong conclusions. The conclusion “infection has been ruled-out” requires appeal to the null disease condition x₀. This conclusion is a stronger than the conclusion “infection has not been ruled-in,” which requires appeal only to the non-null disease condition x₁. Example 2 illustrates diseases that are more finely resolved on a pathophysiologic spectrum. Here, disease is n-ary, where Ω_{X} has n≧2 disease conditions. The number of disease conditions n is subject to revision from improved understanding of the disease, improved tests, or when improved treatment may result. Hypertension is a good example. Its four disease conditions grew out of two—normotensive, and hypertensive.

People get sick, get better, etc. at times that are difficult to predict and for reasons that are not always clear. Accordingly, the state variable for a disease X(t) is a time-dependent random variable, also called a stochastic variable. Thus, the time-evolution of disease in a person is viewed as a random jump process, where a person spends some time in a disease condition then jumps to another disease condition, and so on. Unlike many prior art attempts at processes for quantitative medical diagnosis, the present invention does not require a model the dynamics of the disease state X(t). Instead, embodiments of the present invention provide an estimate of X(t) that depends upon data gathered from the patient through testing. The estimate of disease in a person is called the disease impression.

Here, a medical test is broadly defined as any question, assay, or study that can clarify the condition of a clinical feature (Eq. 1). Preferably, disease conditions of a particular disease are defined to be mutually exclusive and exhaustive. In contrast with other prior art Bayesian approaches, embodiments of the present invention do not require that diseases themselves be mutually exclusive. In fact, the present invention accommodates a reality faced in medical diagnosis that two or more diseases can be completely redundant.

### B. Disease Impression

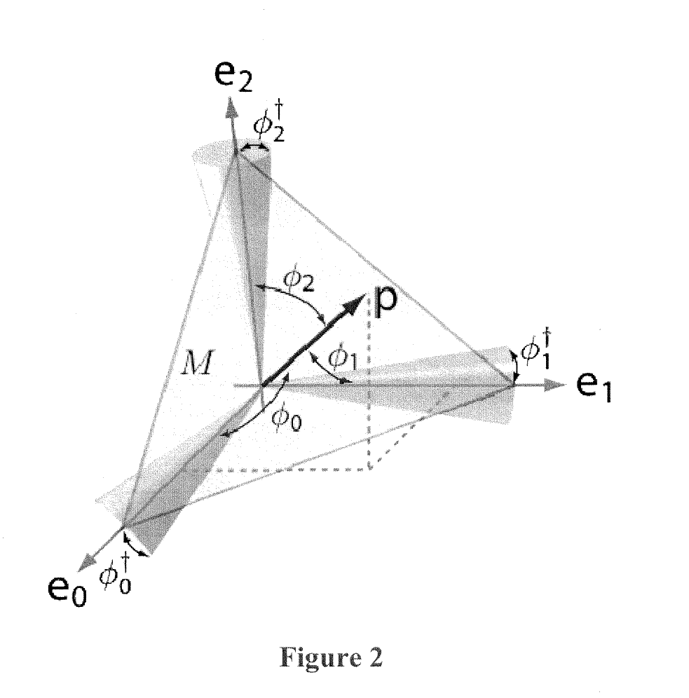

For each disease, we define a discrete probability distribution p=(p₀, . . . , pₙ₋₁), also called a probability mass function or probability density, as the n-tuple of probability elements pᵢ=p(i)=Pr[X=xᵢ], ∀xᵢεΩ_{X}. The element pᵢ is the probability that the state variable X is equal to the disease condition xᵢ. As shown in FIG. 2, it is convenient to visualize the disease impression p as a vector within a generalized n-dimensional Euclidian space R″, also called a Hilbert space, for a disease with n disease conditions x₀, . . . , xₙ₋₁, where each disease condition is represented as an axis. The orthonormal axes e₀, . . . , eₙ₋₁ define this space, which we call the diagnostic space. (While FIG. 2 illustrates a 3-dimensional diagnostic space, which is the largest number of axes conveniently represented on paper, the diagnostic space is not so limited.) Each axis eⱼ=(e₀^{j}, . . . eₙ₋₁^{j}) represents the disease condition xⱼ as a peaked probability distribution, where eᵢ^{j}=δᵢ^{j}. δᵢ^{j}=δ(i−j) is the Kronecker delta that is unity when i=j and zero otherwise. For disease 1 above, there are two axes: not infected, e₀=(1,0), and infected, e₁=(0,1). All vectors within the diagnostic space, including the axes eⱼ, project from the origin to a bounded manifold M⊂ [symbol] ⁿ that is defined by the constraint that all probability elements are real numbers between 0 and 1, pᵢε[0,1], and the constraint that the probability distribution is normalized to 1, |p|=Σ_{{i}}pᵢ=1.

FIG. 2 illustrates the disease impression is a probability distribution p=(p₀, . . . , pₙ₋₁) that rotates in response to test outcomes. The diagnostic space is a probability space whose purpose is similar to that of a phase space in statistical mechanics. The diagnostic space provides a canvas to trace the time-evolution of the disease impression (system) during diagnosis. It also facilitates measures of diagnostic progress. In terms of the diagnostic space, the goal of medical diagnosis is to use the outcomes of tests to rotate the disease impression p until it preferentially aligns with some axis eⱼ. To quantify this statement, consider two vectors a,b εM. [symbol] a,b [symbol] =Σ_{{i}}aᵢbᵢ=a¹bᵢ is the inner product of a and b. ∥a∥= [symbol] is the length of a that adjusts to keep a normalized to 1 (|a|=1). Using these definitions, the angle φⱼ between the vector p and the axis eⱼ (FIG. 2) is obtained from the expression,

[symbol]

p,eⱼ [symbol] =eⱼⁱpᵢ=∥p∥∥eⱼ∥cos φⱼ. (2)

Furthermore, pᵢ=∥p∥cos φᵢ/∥eᵢ∥=∥p∥ cos φᵢ, where ∥p∥=1/Σ_{{j}} cos φⱼ. The length of the disease impression p is a function of all angles φ₀, . . . , φₙ₋₁, which makes each probability element pⱼ—the projection of p onto eⱼ—a function of all angles φ₀,K, φₙ₋₁. Diagnostic progress towards axis eⱼ is expressed more simply and directly in terms of the angle φⱼ rather than the probability element pⱼ. Using the coordinate transform for φᵢ [symbol] pᵢ,

$${p}_{i} = cos {φ}_{i} / {\sum }_{j = 0}^{n - 1} cos {φ}_{j} , \qquad ( 3 )$$

the disease impression p can be equivalently represented as an n-tuple of probability elements (p₀, . . . pₙ₋₁) or as an n-tuple of angles (φ₀, . . . , φₙ₋₁). The angles φᵢ range from 0 to π/2. For convenience, we define normalized angles φ̅ᵢ≡φᵢ/(π/2) that range from 0 to 1. The angle φⱼ^{†} is the threshold of confidence for diagnosing disease condition xⱼ.

### C. Tests

Consider a test whose output is reported as an integer α that assumes some value \hat{α} among m possible outcomes, \hat{α}εΩ_{α}={0,1, . . . , m−1}. For some disease, we will discuss the test where the number of possible test outcomes m of the test equals the number of disease conditions n of the disease. See the Appendix for how to treat the case m≠n. A test Tₖ can inform the disease impressions of multiple diseases D₀, D₁, . . . . Focus on the test Tₖ with respect to disease Dⱼ, and denote the disease-referenced test T_{k←j}. An (n×n)-test has joint probability distribution T given as an n×n matrix with elements Tᵢ^{α}=Pr(α=\hat{α},X=xᵢ)=p(α,i). The element Tᵢ^{α} is the statistical correlation between test outcome \hat{α} and disease condition i. To avoid confusion, we consistently index the outcomes of a test by the Greek letter α and index the disease conditions of a test by the Latin letter i. T is normalized, |T|=Σ_{{α,i}}Tᵢ^{α}=1. Off-diagonal elements Tᵢ^{α}, i≠α that are greater than zero quantify the statistical error, also called the internal noise, of the test. The internal noise of a test is important since it introduces random error into the disease impression p. This makes p a random variable; specifically p is a vector-valued random variable. Note that the state of disease X(t) and the impression p of the state of disease X(t) are different quantities. Both are random variables: X(t) is random because the evolution of disease in a person is a random process, but p is random because it depends on tests that produce error randomly. It is not possible to say when a test will produce its next erroneous outcome. Consequently, internal noise can be described only statistically. A binary test, or (2×2)-test, produces either a negative outcome (α=0) or a positive outcome (α=1). Binary tests are encountered so often that the elements Tᵢ^{α} of the joint probability distribution T have common names: T₀⁰ (true negative), T₀¹ (false positive), T₁⁰ (false negative), T₁¹ (true positive).

The conditional probability distribution of the disease-referenced test T_{k←j} is the likelihood matrix L with elements Lᵢ^{α}=Pr(α=\hat{α}|X=xᵢ)=p(α|i). The element p(α|i) is the probability of obtaining test outcome \hat{α} given that a person is in disease condition xᵢ. The conditional probability distribution L is obtained by renormalizing the joint probability distribution T,

$${L}_{i}^{α} = {T}_{i}^{α} / {\sum }_{α = 0}^{m - 1} {T}_{i}^{α} . \qquad ( 4 )$$

This equation follows from the definition of conditional probability p(α|i)=p(α,i)/p(i), where p(i) is the marginal probability p(i)=Σ_{{α}}p(α,i). The denominator in Eq. 4,

$${r}_{i} = {\sum }_{\{ α \}} {T}_{i}^{α} , \qquad ( 5 )$$

which is the sum over all elements {α} in row i of T, is the prevalence of disease condition xᵢ in the patient population. Eq. 4 guarantees that each row i of the likelihood matrix L is unit normalized, Σ_{{α}}Lᵢ^{α}=1. For a (2×2)-test, the diagonal elements of L have common names: L₁¹ (sensitivity) and L₀⁰ (specificity).

### D. Diagnostic Semantic Web

An ontology is an expression that relates two elements and conveys the meaning of the relationship. The semantic triple, the 3-tuple (subject, object, influence), is the ontology for directionally associating a subject with an object. Having defined the disease impression p of disease Dⱼ, the output α of the disease-referenced test T_{j←k}, and the likelihood matrix L of the disease-referenced test T_{j←k}, the fundamental axiom of allopathic medicine (Eq. 1) may now be expressed quantitatively as a semantic triple (p, α, L). We call this semantic triple a diagnostic triple. The variables of the diagnostic triple and the element that the variable quantifies—disease (box) and its causal influence (arrow) on a test (circle)—are represented as an elementary graph,

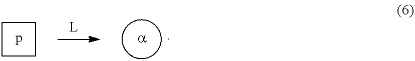

Each variable in the diagnostic triple is a different type. The disease impression p is a probability distribution; the test output α is a random variable; the likelihood L is a matrix.

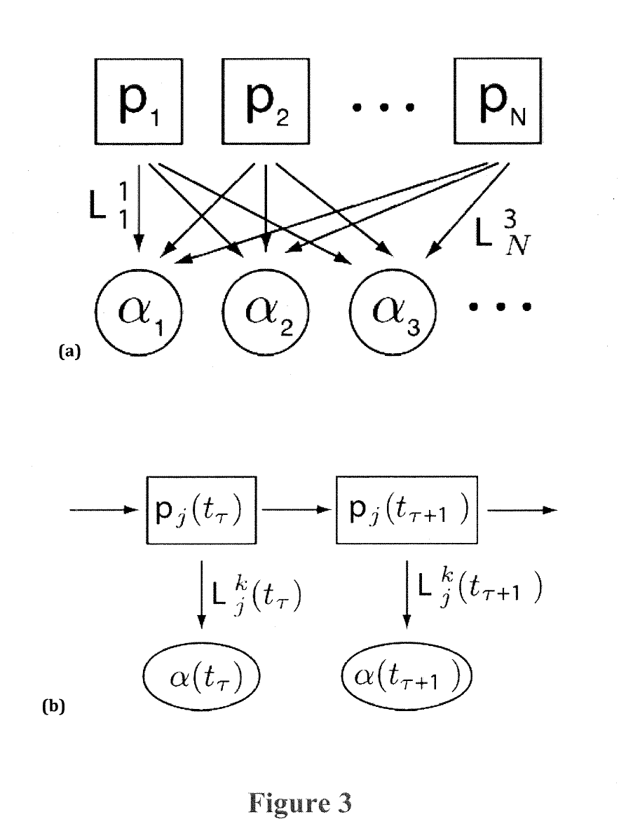

Embodiments of the present invention apply the diagnostic triple to the problem of large-scale diagnosis involving multiple diseases Dⱼ and multiple disease-referenced tests T_{k←j}. Let pⱼ=(p₀^{j}, . . . , pₙ₋₁^{j}) be the disease impression of disease Dⱼ; let Lⱼ^{k} be the likelihood matrix of test T_{k←j}; and let αₖ, be the output of test T_{k←j}. The disease triples for all desired disease/test combinations {(pⱼ, αₖ, Lⱼ^{k})} are assembled as a semantic web (FIG. 3a) that is called the diagnostic web. The collection of likelihood matrices {Lⱼ^{k}} comprises the knowledge base of the diagnostic web. The diagnostic web is dynamic—while the disease layer is fixed, the test layer expands as tests are performed. Two assumptions are implicit: (i) Disease influences test outcomes, but tests do not influence disease. (ii) Each measurement is blind to (not influenced by) the outcomes of previous tests. Assumption (i) overlooks iatrogenic complications in medical testing. Assumption (ii) stipulates that each test measures the person, not other tests. In principle, each test can inform the disease impression of every disease, which makes the diagnostic web dense. In practice, most clinical findings bear on a restricted number of diseases. Thus, the causal influence of most disease/test combinations is weak to none. No causal influence is represented by either deleting the corresponding edge from the diagnostic web or by setting the elements of the edge's likelihood matrix L uniformly equal to 1/n (see below).

### E. Clinical Impression

One might be tempted to define the overall clinical impression of a person's state of health as the set of the disease impressions {pⱼ} for all diseases in a standardized medical lexicon. However, some diseases—derived diseases—are defined as logical operations of elementary diseases. For example, infectious endocarditis D₃ involves both persistent bacteremia D₁ AND cardiac valvular pathology D₂ (D₃=D₁ [symbol] D₂). And for example, a person has chronic obstructive pulmonary disease D₃ if s/he has either emphysema D₁ OR chronic obstructive bronchitis D₂ or both (D₃=D₁ [symbol] D₂). But how do we perform logical Boolean operations (like AND and OR) on probability distributions? Specifically, how do we define AND and OR operations on p₁, p₂, the disease impressions of diseases D₁, D₂ ? The answer should satisfy these desiderata: (i) Boolean operations on diseases D₁, D₂ like AND and OR, are defined with respect to the most severe disease condition xₙ₋₁ of D₁, D₂. (ii) A logical operation [symbol] ε [symbol] of probability distributions must produce a probability distribution, [symbol] :M²→M. (iii) When the disease impression is non-probabilistic (i.e., pᵢ ε{0,1}), the logical operation [symbol] must reduce to the deterministic Boolean algebraic operation. Extending Gödel multi-valued logic satisfies these three desiderata. The conjunction \hat{p}=p₁ [symbol] p₂ is defined,

p₁ [symbol] p₂≡max_{φ}_{ₙ₋₁}{p₁,p₂}.

The disjunction \check{p}=p₁ [symbol] p₂ is defined,

p₁ [symbol] p₂≡min_{φ}_{ₙ₋₁}{p₁,p₂}.

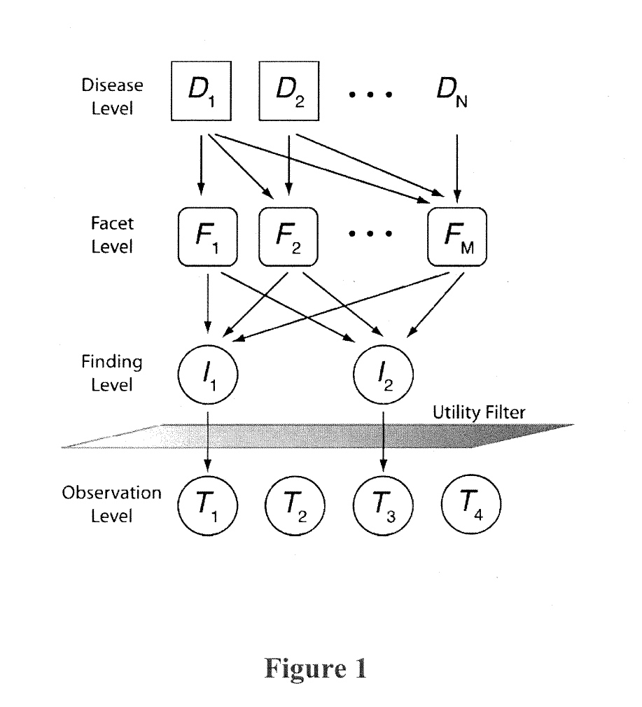

In other words, the conjunction \hat{p} is assigned to whichever disease impression p₁, p₂ has the largest angle φₙ₋₁. The disjunction \check{p} is assigned to whichever disease impression p₁, p₂ has the smallest angle φₙ₋₁. The angle φₙ₋₁ is the angle for the most severe disease condition. The clinical impression {pⱼ, [symbol] } is defined as the set of all elementary diseases and all logically derived diseases. During diagnosis, the clinical impression is updated by independently updating the elementary disease impressions pⱼ then processing the pⱼ to obtain the disease impressions [symbol] of all derived diseases. This representation of disease is consistent with the cognitive model of medical diagnosis, where elementary diseases are called disease facets (FIG. 1).

### III. Results

### A. Inference

The DSW (FIG. 3a) is our model for quantitative medical diagnosis. It can involve all diseases within a standardized medical lexicon and an ordered collection of tests. The DSW is a bipartite directed graph, meaning that it has two classes of nodes with directed links running only between nodes of a different type. Diseases (bottom layer) and tests (top layer) are organized non-hierarchically. The diagnostic web grows as tests T₀, T₁ . . . are added to the bottom layer. Test Tⱼ produces output αⱼ with some outcome \hat{α}ⱼ. The disease impression for each disease (node) evolves

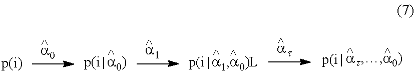

as the outcomes of tests become available. As demonstrated below, when tests are conditionally independent, the disease impression for each disease not only updates as new test outcomes arrive (Eq. 7), but it updates recursively.

Assume tests T₀, T₁, . . . , T_{τ} do not interact so that the outputs α₀, α₁, . . . , α_{τ}, respectively, of the tests are conditionally independent,

p(α_{τ}, . . . ,α₁,α₀|i)=p(α_{τ}|i) . . . p(α₁|i)p(α₀|i). (8)

The disease impression of each disease in the diagnostic web (FIG. 3a) updates recursively in response to the outcomes from tests \hat{α}₀, \hat{α}₁, . . . , \hat{α}_{τ}, from tests T₀, T₁, . . . T_{τ},

$$p ( i \vert {\hat{α}}_{τ} , {\hat{α}}_{τ - 1} , … , {\hat{α}}_{0} ) = \frac{p ( {\hat{α}}_{τ} \vert i ) p ( i \vert {\hat{α}}_{τ - 1} , … , {\hat{α}}_{0} )}{{\sum }_{\{ i \}} p ( {\hat{α}}_{τ} \vert i ) p ( i \vert {\hat{α}}_{τ - 1} , … , {\hat{α}}_{0} )} .$$

Eq. 8 demands that tests be conditionally independent. Whether test findings are conditionally independent is a question about the knowledge base (see discussion, Eq. 13). In other words, Eq. 8 is valid as long as each test provides a measurement of a disease that is blind to the outcomes of previous tests. Thus, the ordered list of tests T₀, T₁ . . . may include a test that has been repeated (e.g., blood cultures in triplicate).

Having proven the foregoing, disease impression becomes a piecewise continuous time-dependent function p(t)=p(t₀),p(t₁), . . . , where p(t_{τ})=p(i|α(t_{τ-1}), . . . , α(t₀)) is the disease impression during the time interval [t_{τ},t_{τ+1}). The time interval Δtᵢ=tᵢ₊₁−tᵢ is not fixed. In our revised notation, α(t_{τ}) is the outcome â of a noisy measurement δ_{α}^{α(t}^{_{τ}}⁾≡δ(α−α(t_{τ})) at the end of the interval [t_{τ}, t_{τ+1})) from the test T (t_{τ}). Now, time is explicit, and the probability elements of each elementary disease impression evolve according to a stochastic difference equation,

$${p}_{i} ( {t}_{τ + 1} ) = \frac{{δ}_{α}^{α ( {t}_{τ} )} {L}_{i}^{α} {p}_{i} ( {t}_{τ} )}{{δ}_{α ( {t}_{τ} )}^{α} {L}_{α}^{i} {p}_{i} ( {t}_{τ} )} = \frac{{L}_{i}^{α ( {t}_{τ} )} {p}_{i} ( {t}_{τ} )}{{L}_{α ( {t}_{τ} )}^{i} {p}_{i} ( {t}_{τ} )} . \qquad ( 9 a )$$

In the final expression, the numerator is the simple product of element Lᵢ^{α(t}^{_{τ}}⁾ of the likelihood matrix Lₖ^{j} and element pᵢ of the disease impression pⱼ of disease Dⱼ. The denominator L_{α(t}_{_{τ}}₎ⁱpᵢ=τ_{{i}}Lᵢ^{α(t}^{_{τ}}⁾pᵢ is the probability of obtaining outcome α(t_{τ}).
Eq. 9a is evolution equation for the disease impression. This and other properties become clearer when Eq. 9a is written in operator form:

p(t_{τ+1})=W(t_{τ})p(t_{τ}) (9b)

where Wᵢ(t_{τ})=Lᵢ^{α(t}^{_{τ}}⁾/L_{α(t}_{_{τ)}}ⁱpᵢ(t_{τ}). Inspection of Eq. 9b and the operator W reveals that updated disease impression p(t_{τ+1}) depends on the current disease impression p(t_{τ}) but not on its history p(t_{τ-1}), p(t_{τ-2}), . . . . Consequently, the disease impression p(t) of each elementary disease evolves as a Markov chain

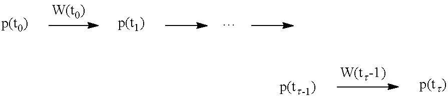

according to the hidden Markov model shown in FIG. 3b.

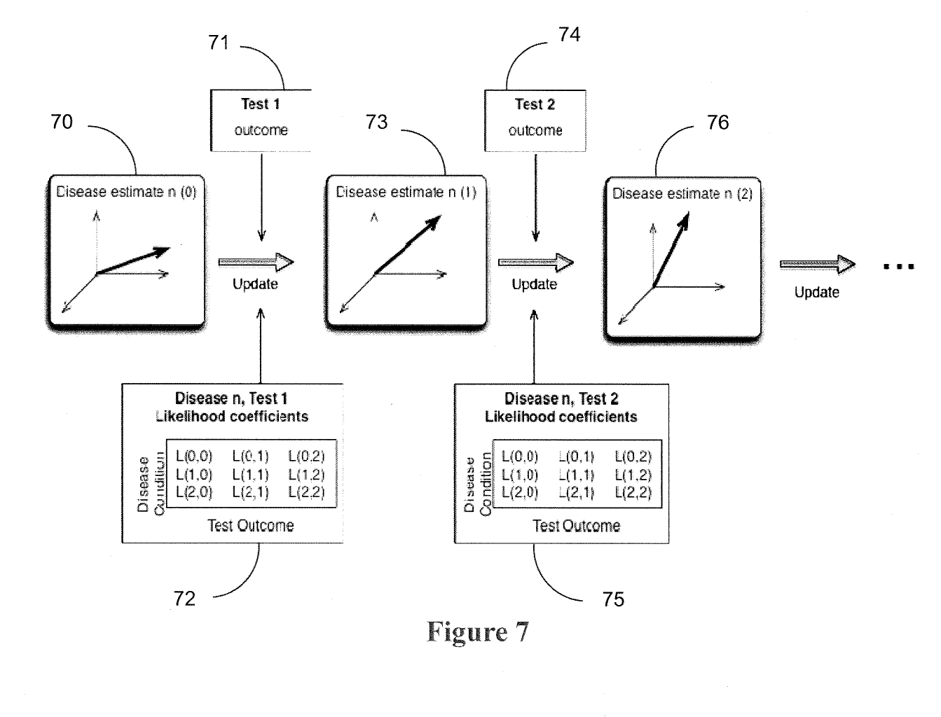

The evolution of disease impression is illustrated graphically in FIG. 7. The process begins with an initial disease estimate or impression n(0) for a Disease n, shown in block 70. Test 1 is run, as shown in block 71, providing a test outcome. The conditional probability of obtaining the test outcome for each condition of Disease n is contained in the likelihood matrix 72. The disease estimate is updated according to Eq. 9 to obtain a new disease estimate n(1), as shown in block 73. The process is repeated with Test 2, shown in block 74, and the likelihood matrix 75 for Disease n and Test 2, to obtain Disease estimate n(2), in block 76, and so on for x iterations, until a threshold of confidence is achieved that the disease estimate n(1+x) corresponds to one of the disease conditions of Disease n.

Embodiments of the present invention, therefore, allow the simultaneous diagnosis of multiple diseases—large-scale medical diagnosis—via solving a parallel stochastic filtering problem, where the disease impressions of all elementary diseases are independently updated as test outcomes α(tₛ) arrive. Eq. 9 provides the updating, but the process must begin with some initial condition p(t₀). One may assume complete ignorance and adopt an equivocal disease impression—the uniform distribution pᵢ(t₀)=1/n. A better alternative is to use the prevalence of disease in the population demographic, that is the age-classified, gender-classified, and if appropriate geography- and racial/ethnic-classified, population from which the patient comes, pᵢ(t₀)=rᵢ, where rᵢ is given by Eq. 5.

### B. Prediction

In addition to estimating the state of disease from the outcomes of tests, it is useful to predict how a test could change the clinical impression. Prediction is important when designing a diagnostic strategy since the predicted change in the clinical impression is the anticipated diagnostic benefit in a cost/benefit analysis of test options. Bearing in mind that tests are subject to random error, the desired quantity is the most probable effect that a test will have on the clinical impression. The following example is of a binary disease, but the method illustrated is applicable to diseases of any dimension.

Begin by assuming that a person is in some disease condition X=xⱼ, or, equivalently, p*=eⱼ. If the goal is to rule-in the disease (we think that X=x₁) then p*=(0,1). If the goal is to rule-out the disease (we think that X=x₀) then p*=(1,0). For a person who is known or assumed to be in disease condition p*, the probability of obtaining result α of some test with likelihood matrix L is the inner product of L and p*, ρ_{α}= [symbol] L,p* [symbol] =L_{α}ⁱp*ᵢ. The unit-normalized rows of Lᵢ^{α} guarantee that ρ_{α} is a probability distribution, so ρ_{α} can be represented as a point on the manifold M (FIG. 2) (p_{α}εM). The expected (or average) time-projected (projected) disease impression E(p(t_{τ+1}))= [symbol] p(t_{τ+1}) [symbol] is the inner product [symbol] p(t_{τ+1}|α),ρ_{α} [symbol] . After substituting Eq. 9, we obtain

$$〈 {p}_{i} ( {t}_{τ + 1} ) 〉 = {L}_{i}^{α} ( \frac{{L}_{α}^{j} {p}_{j}^{*} ( {t}_{τ} )}{{L}_{α}^{k} {p}_{k} ( {t}_{τ} )} ) {p}_{i} ( {t}_{τ} ) . \qquad ( 10 )$$

The term in parentheses is a vector over α, which, when left-multiplied by Lᵢ^{α} produces a vector over i. Eq. 10 provides the expected projected disease impression after performing a test T(t_{τ}) for a person in disease condition p*. To predict behavior beyond the average, one simulates the measurement δ_{α}^{\hat{α}(t)} by Monte Carlo sampling from ρ_{α} then applies the measurement to Eq. 9. This approach provides higher moments of the distribution p(p(t_{τ+1})) of the projected disease impression. Since Eq. 10 depends on pᵢ(t_{τ}) nonlinearly, exact prediction with two or more tests requires calculation of the full distribution p(p(t)). But, making the approximation pᵢ(t_{τ})≈ [symbol] pᵢ(t_{τ}) [symbol] on the right hand side of Eq. 10, we obtain the expression

$$〈 {p}_{i} ( {t}_{τ + 1} ) 〉 \approx {L}_{i}^{α} ( \frac{{L}_{α}^{j} {p}_{j}^{*} ( {t}_{τ} )}{{L}_{α}^{k} 〈 {p}_{i} ( {t}_{τ} ) 〉} ) 〈 {p}_{i} ( {t}_{τ} ) 〉 \qquad ( 11 )$$

to recursively to calculate, approximately, the expected disease impression after a succession of tests. In terms of the diagnostic space, the expected projected disease impression [symbol] p(t_{τ}) [symbol] is a point on the manifold M, while the full distribution p(p(t_{τ})) of projected disease impression is a probability density on M.

### C. Miscellaneous Properties

We conclude methods development by answering two important questions. First, we ask, “What is the average predicted long-term (stationary) disease estimate [symbol] p^{s} [symbol] = [symbol] p(t_{∞}) [symbol] after repeatedly testing a person whose disease state is the disease condition p*?” For a person in the time-independent disease condition p*, applying Eq. 10 an infinite number of times causes the average disease estimate to either not change, [symbol] p^{s} [symbol] =p(0) (trivial case, where tests are non-informative, Lᵢ^{α}=1/n), or to converge on the disease-distribution, [symbol] p^{s} [symbol] =p*.

Second, we ask, “Does the disease estimate p(t_{τ}) depend on the order that test outcomes α(t₀),α(t₁), . . . , α(t_{τ-1}) are applied to propagate p(t)?” Eq. 9 satisfies the commutative property, p(i|α₁,α₂)=p(i|α₂,α₁). By extension, the inferred disease impression p(t_{τ}) depends on the accumulated test outcomes {α(t₀),α(t₁), . . . , α(t_{τ-1})} but not their sequence. Therefore, when tests are conditionally independent (Eq. 8), the final disease impression is independent of the diagnostic path. In other words, the disease impression depends only on the starting estimate and the accumulated evidence.

### D. Examples

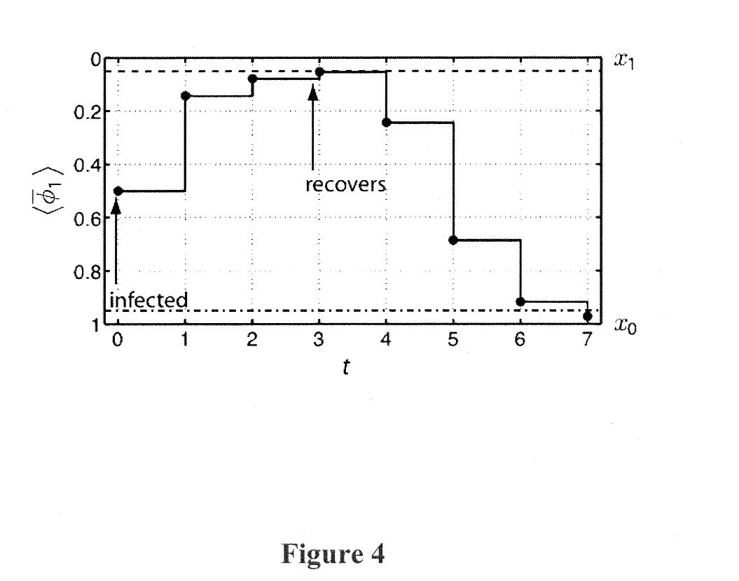

The method of prediction was examined with an example of the diagnosis of infection (example 1 above). The example involved a patient who was infected at times t₀, t₁, t₂ (p*=e₁) then recovered between times t₂ and t₃ (p*=e₀) and remained uninfected. This example, where the patient is initially infected then recovers, was chosen to illustrate how an embodiment of the method of the present invention perform temporal reasoning—reasoning as the patient condition itself changes, also called patient dynamics. FIG. 4 shows the expected time course of the diagnosis. The trajectory of the expected disease impression [symbol] p(t) [symbol] was calculated using Eq. 11 for the repeated application of a single test. Disease conditions: x₁ (infected), x₀ (non-infected). Expected normalized angle to axis e₁, φ̅₁(circles, solid line) from Eqs. 10 and 2, diagnostic thresholds: φ̅₁^{\} (dashed line), 1− φ̅₁^{\} (dash dotted line). Note: [symbol] φ [symbol] =1− [symbol] φ̅₁ [symbol] . At times t≦t₂, X=x₁ (p*=e₁). At times t≦t₃, X=x₀ (p*=e₀). Simulation parameters: specificity, L₀⁰=0.98; sensitivity, L₁¹=0.80; initial estimate, p(t₀)=(0.5, 0.5); diagnostic thresholds, φ̅₀^{†}= φ̅₁^{\=}0.05. Regardless of whether the patient is infected or non-infected, the expected disease impression asymptotically approaches the disease condition p*(t_{τ}) at the time the test was performed. Thus, the DSW is capable of treating patient dynamics.

When any angle φⱼ becomes smaller than its associated threshold angle φⱼ^{†} (FIG. 2), a diagnosis of disease condition xⱼ is made

Diagnosis: xⱼ if φⱼ<φⱼ†

with confidence pⱼ. In the example shown in FIG. 4, the patient remains undiagnosed until time t₇ when φ₀(t₇) falls below φ₀^{†}. Geometrically, the threshold for diagnosis is a bounded arc with points pⱼ^{†}, defined by the intersection of the cone with fixed φⱼ^{†} and the bounded manifold M, as shown in FIG. 2. The probability element pⱼ is not constant on this arc, so the confidence in a diagnosis will depend somewhat on where the diagnostic threshold arc is crossed.

### E. Measures of Diagnostic Progress

Some think of medical diagnosis as an attempt to reduce uncertainty in the mind of the clinician, where reduced uncertainty is quantified as a loss of Shannon entropy or, equivalently, the gain of Shannon information. The example in FIG. 4 shows why diagnostic progress should not be equated with the gain of information. After the patient recovers, to track the new disease-condition x₀ the disease impression must rotate away from axis e₁ towards axis e₀. This action involves the transient loss of information, where information is defined as negative relative entropy −D_{KL}[p(t_{τ+1})Å [symbol] (t_{τ})]. It is preferable, and accurate, to simply view medical diagnosis as a locking-on of the disease impression p(t) to the time-dependent disease condition X(t) of the patient.

The measure of progress towards the diagnosis of disease condition xⱼ should quantify progress of the disease impression towards axis eⱼ. Furthermore, diagnostic progress should not be influenced by changes in the length of the disease impression p that are required to keep the disease impression unit-normalized, |p|=1. Two measures for quantifying diagnostic progress are appropriate: (i) the angular change in the disease impression Δφⱼ(t_{τ})=φⱼ(t_{τ+1})−φⱼ(t₉₆) due to the test outcome α(t_{τ}) and (ii) the rotational strain

Úⱼ(t_{τ})=−Δφⱼ(t_{τ})/φⱼ(t_{τ}) (12)

due to the test outcome α(t_{τ}). Rotational strain is dimensionless and bounded, Úⱼε[0,1], with Úⱼ=0 being no progress and Úⱼ=1 being maximum progress. The predicted utility of a test can be measured using the average predicted angular strain [symbol] Úⱼ(t_{τ}) [symbol] , which is based on the average projected disease impression (Eq. 10 or 11).

A practical measure of predicted test utility is the expected number of times NNDᵢ (number needed to diagnose) that a test must be repeated to diagnose disease condition xᵢ given the current disease impression p(t_{τ}). A test with low NNDᵢ is more powerful for diagnosing disease condition xᵢ than a test with a high NNDᵢ, NNDᵢ depends on three factors: (i) the likelihood matrix Lᵢ^{α} of the test, (ii) the threshold for diagnosis φᵢ^{†}, and (iii) the current disease impression p(t_{τ}). NNDᵢ is not a constant; its dependence on p(t_{τ}) makes NND, a time-dependent function NNDᵢ(t_{τ}). For the example in FIG. 4, NND₁(t₀)=4 and NND₀(t₃)=4.

### F. Effects of Non-Linearity

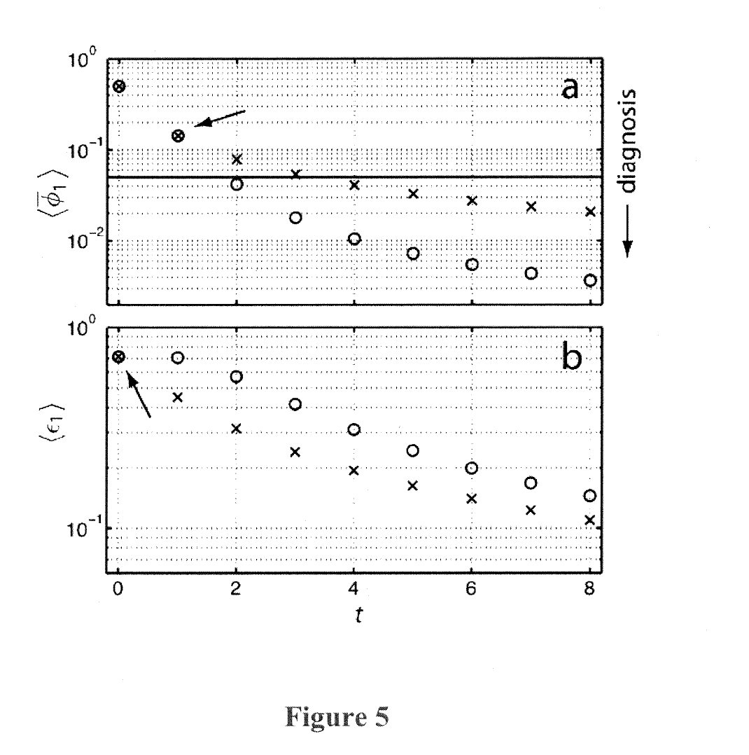

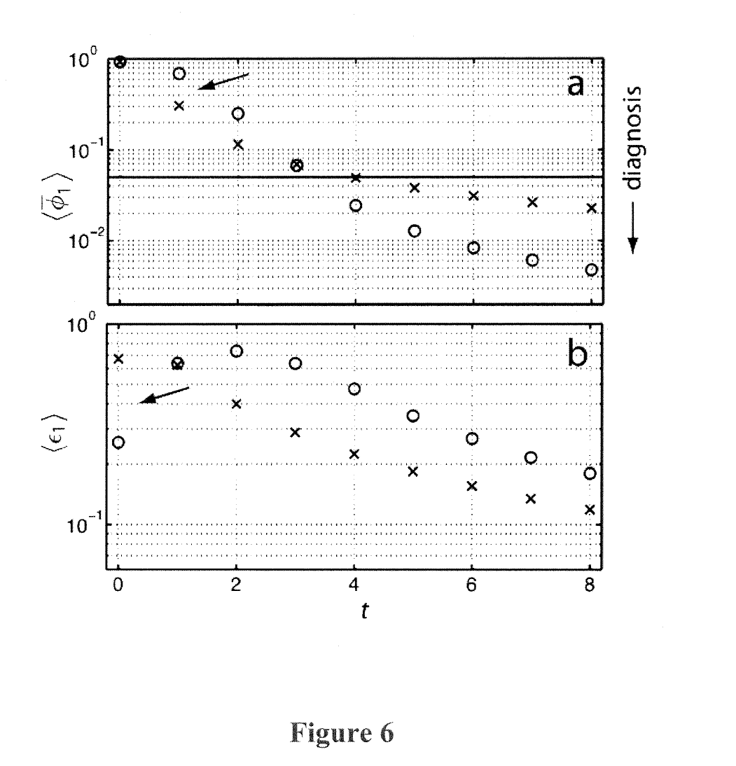

A rule in medical diagnosis is that high sensitivity tests should be used to rule-in disease (diagnose disease condition x₁) while high specificity tests should be used to rule-out disease (diagnose disease condition x₀). The example shown in FIGS. 5 and 6 show that this rule does not hold universally. The rule applies when one is attempting to diagnose a disease condition that is already favored by the current disease impression. FIGS. 5 and 6 compare the utility of two tests T₁, T₂ to rule-in disease, that is, to diagnose infection in a patient who is infected, p*=e₁. T₁ (circles): specificity L₀⁰=0.80, sensitivity L₁¹=0.98. T₂ (crosses): specificity L₀⁰=0.98, sensitivity L₁¹=0.80. Initial disease impression p(t₀)=(0.5, 0.5) is equivocal. (a) Average normalized angle [symbol] φ₁ [symbol] (Eq. 2) from the average projected disease impression (Eq. 10). Threshold for positive diagnosis: φ̅₁^{\}=0.05 (solid line). (b) Average predicted angular strain [symbol] Úⱼ [symbol] (Eq. 11) by the test. Tests T₁ and T₂ have the same accuracy, but test T₁ has high sensitivity (L₁¹=0.98), while T₂ has low sensitivity (L₁¹, =0.80). The arrows show where the rule breaks down. The accuracy of test Tᵢ is defined as the dimension-normalized trace of its likelihood matrix, tr(Lᵢ)/n. Since tests T₁ and T₂ have the same accuracy, tr(L₁)=tr (L₂)=0.80+0.98. As expected from the rule, when the disease impression favors disease condition x₁, the more sensitive test T₁ has greater utility to rule-in disease. However, when the disease impression is equivocal (p=(0.5, 0.5)), tests T₁ and T₂ have equal utility to rule-in disease, despite T₁ being a more sensitive test. The parameters for the example in FIG. 6 are the same as those in FIG. 5, except that the initial disease impression, p(t₀)=(0.9, 0.1), favors non-infection. Arrows show where the rule breaks down. Paradoxically, when the disease impression favors the disease-free disease condition x₀, the less sensitive test T₂ has greater utility to rule-in disease. We conclude that test utility depends strongly on the current disease impression. This is due to the non-linear dependence of the inferred disease impression on the current disease impression (Eqs. 9, 10, 11). To reliably assess the utility of a test, Eq. 9 should be evaluated.

### III. Advantages

As shown above, large-scale medical diagnosis—the simultaneous diagnosis of all diseases in the standardized medical lexicon—is modeled as a Diagnostic Semantic Web (DSW). The DSW is a special kind of bipartite semantic web with a layer of diseases of fixed size and a layer of tests that grows as tests are performed (FIG. 3a). In this approach to medical diagnosis, the disease impression—the clinician's impression of the state of a patient with respect to some disease—is the elementary object of interest. The disease impression associated with each elementary disease evolves according to a hidden Markov model (FIG. 3b) in response to the outcome of each new test. The clinical impression of a patient is the collection of disease impressions of all diseases. In this way, embodiments of the present invention provide solutions to large-scale medical diagnosis as a stochastic filtering problem that is trivially parallel. Stochasticity in each disease impression is caused by the random error in tests. The main result are two methods including algorithms that support the two essential tasks of medical diagnosis: inference and prediction. The first algorithm (Eq. 9) produces the updated disease impression given a new clinical finding. The second algorithm (Eq. 11) quantifies the expected benefit of a test to further the diagnosis of disease. The algorithms use a common knowledge base of population-based statistical data.

Each disease preferably constitutes an exhaustive set of n≦2 non-overlapping disease conditions that are arranged in order of increasing severity. The disease-free condition is preferably included as the first disease condition. Thus, diseases with any number of disease conditions are treated. A test is defined broadly as any time-resolved source of clinical information. This includes information from the history (e.g., family history, social history, review of systems) and the physical (e.g. physical exam, preliminary laboratory results, imaging studies). The disease impression is represented as a vector that is confined to a manifold within a Hilbert space (FIG. 2) whose axes are the possible conditions of the disease. Diagnostic progress with respect to a disease is visualized and quantified as the rotation of the disease impression towards some disease condition axis. A diagnosis is made when the angle between the disease impression and the axis of some disease condition is less than a pre-defined threshold. Confidence in a diagnosis is the projection of the disease impression onto the axis of the diagnosed disease condition.

### A. Probabilistic Models of Medical Inference

Prior art approaches to quantifying medical diagnosis have been unable to provide a solution that is computationally tractable and implements the three characteristics of medical diagnosis. Medical diagnosis is a dynamic real-time process; the clinician's disease impression of disease is probabilistic; and the disease impression is updated given new clinical findings. Early models of medical diagnosis supported deterministic reasoning. Deterministic models included hypothetico-deductive models, decision trees, and heuristic pattern recognition models. Deterministic models were adapted to support probabilistic reasoning. Heuristic models were extended using confirmation theory and as Bayesian networks, and decision trees were extended using fuzzy set theory. Among these, Bayesian networks seemed most promising, but solving for the probabilities of interest is not feasible for large systems, whether the solution is exact or approximate. Modified (or reduced) networks of the parent Bayesian network were sought. The latter were, essentially, attempts to discover alternative probability-based models for computer-aided medical diagnosis that were computationally tractable and also treated the temporal, iterative, and adaptive nature of diagnosis.

Historically, large-scale medical diagnosis has been viewed as a pattern recognition problem. Large-scale medical diagnosis has been modeled (treated computationally) as a heuristic Bayesian problem, as a Bayesian network, using heuristic Bayesian sequential decision theory, and, most recently, using Dynamic Bayesian network (DBN)-based sequential decision theory.

Sequential diagnosis—Many approaches in computer-aided diagnosis analyze clinical information retrospectively. An alternative strategy, which follows the normal clinical workflow, is to perform a sequential diagnosis, considering new test results as they arrive. Gorry and Barnett recognized the potential to apply Bayes rule recursively to propagate belief. DBN address the temporal nature of diagnostic reasoning by replicating a stationary Bayesian network, also called a belief network, and adding temporal transitions between the stationary Bayesian networks. DBN, however, are not practical. The amount of computing time that is needed to exactly calculate the marginal probability density for each disease limits the size of Bayesian networks in medical diagnosis. Approximate methods reduce calculation times, but approximation methods are limited to Bayesian networks that treat binary diseases.

Here, by treating medical diagnosis as a filtering problem, the number of disease conditions that can be treated is practically unlimited. The disease impression of each disease is updated independently, making medical diagnosis trivially parallelizable. Others have addressed the limited time-scope of a test result (e.g., a person's age is accurate for only one year) by recasting the fixed conditional probabilities in the knowledge base as time-dependent quantities. When medical diagnosis is viewed as a filtering problem, such manipulation is unnecessary. Any change in the result of a test is viewed as a new outcome of the test, and the disease impression is updated using either Eq. 9 or, in certain cases, using Eq. S5 (see below). In this way, the limited time-scope of a test output is treated automatically, and repeated tests are treated seamlessly.

Also, the time-evolution of a dynamic network, be it a dynamic Bayesian network, Markov network, Markov random field, artificial neural network, or semantic web, is governed by a discrete-time (Eq. 9b) or continuous-time master equation. The properties of the master equation depend on the properties of the propagation operator W. The propagation operator for the DSW has an attractive property: it depends only on the knowledge base, which is stationary, and the current disease impression p(t_{τ}). Consequently, the time-evolution of the disease impression is a non-linear (in p) Markov chain. W is a time-dependent operator since it depends on the current disease impression.

The non-linear dependence on the disease impression can produce counter-intuitive results. A rule in medical diagnosis is that high sensitivity tests should be used to rule-in disease while high specificity tests should be used to rule-out disease. Due to the non-linear dependence on the disease impression, we showed above that this rule does not hold universally. Eqs. 10 and 11 provide the average predicted the utility of a test to further the diagnosis of some disease. When crafting a diagnostic strategy, the predicted utility of a test is the expected benefit in a cost-benefit analysis of test options. It may be helpful to re-label the cost-benefit analysis as a cost-benefit-quality analysis, where cost means monetary cost and other factors like the possibility of a missed diagnosis are categorized under benefit.

Structural differences in graphical models—differences between the present invention and the prior art create important structural differences between the DSW and other graphical models of medical diagnosis, which include the Markov network, the Markov random field, the artificial neural network, and the Bayesian network. Semantic webs, Markov random fields, and Markov networks have numerical expressions associated with the edges of their graph. In a Markov network, the expression is a scalar that governs the probability that the system will transition along the edge. In the Markov random field, a potential energy function is defined for sets of nodes. In the DSW, the edge-associated expression is the matrix L′, that quantifies the statistical correlation between the outcomes of a test and conditions of a disease. Bayesian networks and artificial neural networks have no parameters associated with their edges. In Bayesian networks, conditional probabilities are absorbed into the nodes. In artificial neural networks, non-linear activation functions govern the state of nodes.

For convenience, we first list features that distinguish certain embodiments of the present invention from the prior art; then each is discussed. (i) Tests are assumed to be error prone. (ii) Diseases are n-ary: State of a disease is one of n≦2 mutually exclusive conditions, ordered in increasing severity. The null disease condition (n=0) is included. (iii) Diseases need not be mutually exclusive; they may even be redundant. (iv) Knowledge base relies on coarse-graining, rather than causal independence. (v) Probability distributions are defined locally for a particular disease. The estimate of the state of each disease is the probability distribution. (vi) Equitable treatment of positive and negative findings.

Tests are error-prone—Prior art methods typically assume that no errors are made in performing tests and do not make allowance for possible error. As a result, inferred (posterior) probabilities are regarded as subjective measures of belief. Conclusions are drawn from the inferred probabilities though Bayesian hypothesis testing or some kind of decision theoretic optimization. Here, the present invention utilizes the traditional frequencist interpretation of probability and allows for tests to be error prone.

Diseases are n-ary—-Previous Bayesian treatments required diseases to be binary. Positive findings and negative findings in these systems require unique nodes. There is a big difference between the two statements (i) aortic dissection has not been diagnosed and (ii) aortic dissection has been ruled-out.

Diseases need not be mutually exclusive—Other Bayesian approaches require diseases to be mutually exclusive; i.e., all diseases must be defined so that there is no overlap among them. This is an unrealistic assumption. Consider: How does one define as mutually exclusive diseases disseminated intravascular coagulation, HELLP syndrome, hypertension, pre-eclampsia, nephritic syndrome, thrombocytopenia? A woman with HELLP syndrome has all the above. Solving the joint probability distribution is NP-hard. The probability of the patient being disease-free with respect to some disease is a complicated function of the global distribution function. Here, diseases need not be mutually exclusive. We showed above how to the present invention is capable of treating diseases that are defined as logical operations of more elementary diseases.

Local vs. global probability distribution—Previous approaches regard probability as a global quantity—a (joint) probability distribution is defined over the positive disease conditions of all diseases. Treating probability as a global quantity limits how diseases must be defined, makes the problem computationally intractable, and creates conceptual problems that produce wrong conclusions.

Tests are Error-prone—The reader may be left with the impression that the diagnostic uncertainty is fundamentally due to incomplete information about a patient. Here, diagnostic uncertainty is fundamentally due to uncertainty stemming from the fact that tests are error-prone. A property of random error is that you never know when errors will appear. It is possible that a test with a specificity of 99% could produce three false positives in a row (on the same patient), which lead to an incorrect diagnosis and incorrect treatment that damaged the patient. These cases will occur because tests are fundamentally error-prone. There is nothing that can be done. The present invention accepts this fact of clinical life.

Equitable treatment of positive and negative findings—Previous models treated diseases as positive quantities—as in, you have a disease as a hypothesis. To claim that one did not have a disease (i.e. to rule-out a disease) was treated as the compliment of the hypothesis. This is a problem for two reasons. First, taking the compliment is non trivial. Second, one can only treat binary diseases; n-ary diseases are not accessible.

Local vs. global—Here, we regard probability as a local quantity—each disease has its own probability distribution. The summary impression of a patient, the clinical impression, is the set of disease impressions for each disease in a standard medical lexicon. Diseases need not be mutually exclusive. In the extreme case of diseases that are completely redundant, one simple obtains identical disease impressions for the redundantly defined diseases, as it should be.

### B. Knowledge Base

One limiting factor in computer-aided medical diagnosis is the knowledge base for diagnosis, which consists of the statistical correlations (likelihood coefficients) Lᵢ^{α}=p(α|i) for each disease-test pair. In some cases, the Lᵢ^{α} are available in the medical literature. In other cases, the Lᵢ^{α} can be obtained from the analysis of historical data. It is noteworthy that the likelihood coefficients in the literature suffer two limitations. First, the Lᵢ^{α} are coarse-grained, also called marginalized, versions of the more finely resolved joint conditional probabilities p(α|i,j) of a test T with respect to two diseases Dᵢ, Dⱼ. The marginal probability density p(α|i) for a test T and disease Dᵢ is obtained by averaging over all disease conditions of the co-morbid disease Dⱼ,

p(α|i)=Σp(α|j,i)p(j|i). (13)

Eq. 13 is easily generalized for multiple co-morbid diseases {Dⱼ}≠Dᵢ. If the joint conditional probabilities p(α|i,j) are available then the marginal likelihoods p(α|i) can be calculated during diagnosis from the joint conditional density p(α|i,j) and the current disease impressions p(i), p(j) of diseases Dᵢ, Dⱼ using

$$p ( α \vert i ) = \frac{1}{p ( i )} {\sum }_{\{ j \}} p ( α \vert i , j ) p ( i \vert j ) p ( j ) . \qquad ( 14 )$$

Eq. 14 is derived in the Appendix. A second limitation of the marginal likelihoods in the literature is that they may not reflect the conditions for a particular location/region. Despite their limitations, marginal likelihoods p(α|i) comprise the population-based evidence in evidence-based medical diagnosis. These coefficients provide a starting knowledge base for conducting quantitative medical diagnosis. Expert diagnosticians will distinguish themselves by supplying joint conditional probabilities like p(α|i,j) and p(α₂|α₁,i) that are derived from experience. Expert diagnosticians will also use likelihood coefficients that are appropriate for the location, time, and circumstance. One embodiment of the system of the present invention allows a clinician to adjust elements of the likelihood matrices, and also supply joint conditional probabilities, based on experience.

By employing a coarse-grained knowledge base, we avoid the noisy-OR assumption and its limitations. In the noisy-OR assumption, causal independence is assumed between diseases on findings.

### C. Conditional Independence of Tests

The major simplifying assumption in Bayesian approaches in medical diagnosis, including in the discussion above, is that tests T₁, T₂, . . . , T_{τ} are conditionally independent (Eq. 8). The conditional independence assumption is a zero-order approximation that can be replaced with higher-order approximations where appropriate. Through an example, we clarify the meaning of the conditional independence of tests. Then we consider what if tests are not conditionally independent. We show how to treat tests that are pair-wise conditionally dependent (so-called first-order approximation).

Consider the cause of elevated body temperature—the outcome of the test T₂. One can claim that elevated body temperature is more likely to be caused by infection (disease Dᵢ) than, say, hyperthyroidism (disease D₂) because a cardiac echocardiogram (test Tᵢ) showed a valvular vegetation, which suggests that the patient has infective endocarditis. Here, the clinician reasons correctly that the interpretation of fever (test T₂) should depend on the outcome of the echocardiogram (previous test T₁). But what does “interpret test T₂” mean? The interpretation of test T₂ means drawing an inference about a patient's disease condition based on the outcome of test T₂ and, perhaps, also on the outcome of a previous test T₁−p(i|α₂,α₁). While true, this statement about dependencies when inferring the state of disease from two tests is entirely separate from statements about whether two tests independently measure the state of disease in a person, namely that “tests T₁ and T₂ are conditionally independent” p(α₂,α₁|i)p(α₁|i). Another way of stating that test T₂ is conditionally independent from test T₁ (Eq. 8) is to say that the outcome of test T₂ depends on the disease condition of the patient but does not depend on the outcome of some other test T₁, p(α₂|α₁,i)=p(α₂|i)}. See the Appendix for proof that this equation (Eq. S1) is equivalent to Eq. 8. A counter example: If a pathologist based his reading of a biopsy on some other study (say an imaging study) instead of the sample at hand, this would be fraudulent result and would flagrantly violate of Eq, S1 (and hence Eq. 8). Thus, the conditional independence assumption can be regarded as an assumption of honesty—tests can't peek at each other; they offer independent “readings” of the patient.

The inference algorithm (Eq. 9) and prediction algorithm (Eq. 10) can be modified in a straightforward manner to accommodate cases where Eq. 8 or, equivalently, Eq. S1 does not hold. When Eq. S1 is not valid, one uses a propagation operator W that depends on a previous test, p(α₂|α₁,i), instead of an operator that does not depend on other tests, p(α₂|i). Now, the disease impression evolves according to a lesser update rule,

$$p ( i \vert {α}_{2} , {α}_{1} ) = \frac{p ( {α}_{2} \vert i , {α}_{1} )}{{\sum }_{\{ i \}} p ( {α}_{2} \vert i , {α}_{1} ) p ( i \vert {α}_{1} )} p ( i \vert {α}_{1} ) .$$

that is derived in the appendix (Eq. A5). This equation deals explicitly with the pair-wise conditional dependence of two tests.

In some cases, the temporal sequence of observations can provide strong clues for a diagnosis. For example, abdominal pain T₁ followed by gastrointestinal distress T₂ suggests appendicitis, while gastrointestinal distress followed by pain suggests gastroenteritis, p(α₂|i,α₁)≠P(α₁|i,α₂). Medical diagnosis remains Markovian under the first-order approximation (when pair-wise conditional dependencies are admitted), but the commutative property is lost. Whether it is feasible to populate a knowledge base of conditional probabilities p(α₂|i,α₁) for pair-wise conditionally dependent tests is a separate issue.

In certain extreme cases, two tests are completely conditionally dependent, meaning that the second test is redundant of the first. A good example would be repeatedly asking (testing) a person's age or gender. For redundant tests, which tend to appear in the patient history rather than the physical exam, collecting data more than once is useless. Here,

p(αₛ, . . . , α₁,α₀|i)=p(α₀|i),

where α₀, α₁, . . . , αₛ are the outputs of a test that has been repeated s times. For redundant tests, we define a switchable likelihood matrix Lᵢ^{α} (t)=Lᵢ^{α}+F(t)(1/n−Lᵢ^{α}) where F(t) is a Boolean flag that is set, 0→1, once the outcome of this test has been used to update the disease impression. When F(t) is 1, the outcome of a repeated test is non-informative since a test with all likelihood coefficients set equal to 1/n is non-informative. This is verified by direct substitution into Eq. 9.

### IV. Implementation

It will be understood by those skilled in the art that the foregoing may implemented in a computer-based medical information and record system. Such a system may consist of a single computer or comprise multiple computers in communication over a network, including a local area network, a wide area network, the Internet, or any combination of the foregoing.

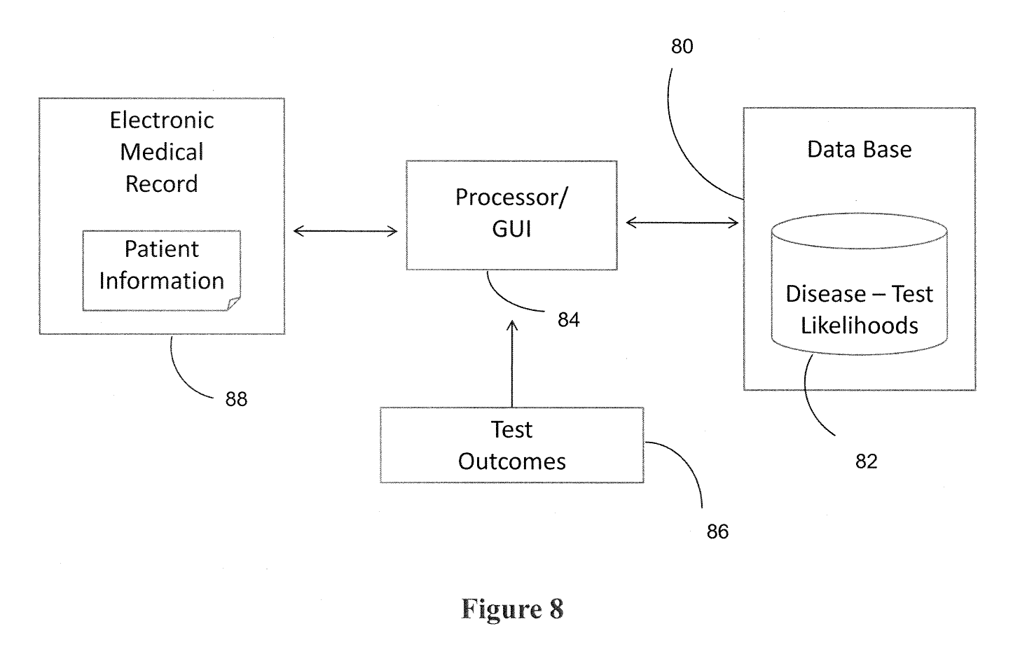

As shown in FIG. 8, the system includes, in any implementation, a medical knowledge base or database 80 stored in a computer readable memory comprising the likelihood matrices 82 described herein, that is, a matrix having as its elements the conditional probability distribution of the conditional probability of obtaining each test outcome given each disease condition of each disease of interest. The system also includes at least one processor 84, preferably in operable communication with a graphical user interface, in communication with the database 80 and with a user (typically a clinician), the processor having a computer readable memory storing instructions executable by the processor for performing the methods described herein. The processor may receive test outcomes 86 directly from a user or it may retrieve them from an electronic medical record 88 accessible to the processor from its own memory, or stored in another computer system with which it is in communication over a network. The processor 84 may also receive test outcomes 86 directly from medical or diagnostic testing equipment in communication with the processor, either directly or over a network. The computer readable memory may be any medium now known or hereafter developed capable of storing information readable by a computer, including, by way of example, hard drives, flash memory, random access memory, optical drives and storage media, or the like. In a typical implementation, the processor 84 is part of a computer system comprising a GUI having input and output devices, such as a keyboard, touch screen, voice recognition, and display panel, or other such input and output devices for communicating with a human user as may be hereafter developed.

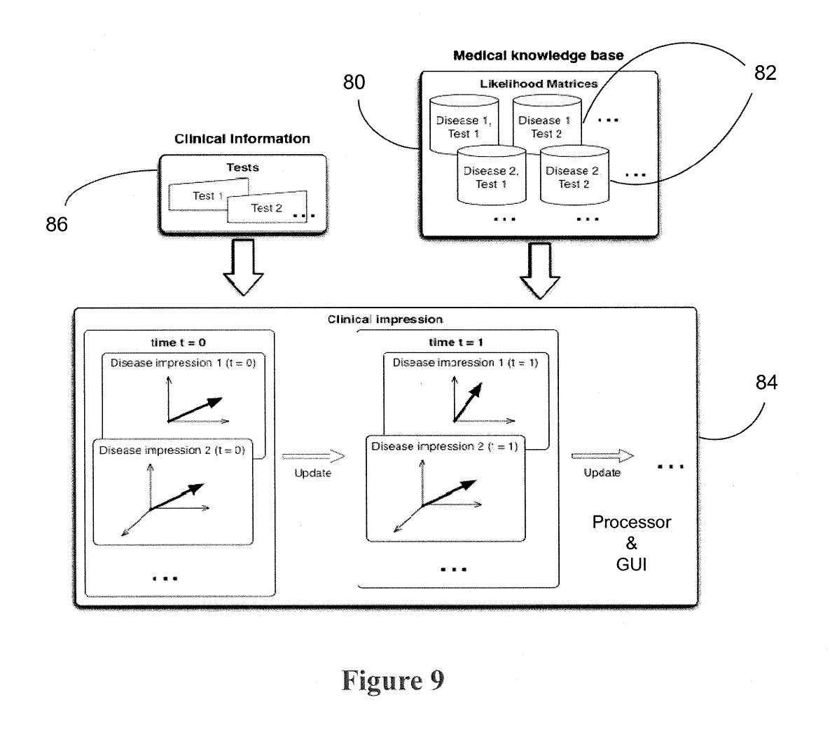

FIG. 9 is an alternative view of an embodiment of the system of the present invention, with the medical knowledge base or database 80 comprising likelihood matrices 82 in communication with a processor and GUI 84, which receives clinical information in the form of tests 86. As shown, the processor 84 executes the process of providing the clinical impression by updating a first disease impression 88 and a second disease impression 89 over time, in response to the received test outcomes 86 and the corresponding conditional probabilities retrieved from the database 80.

The tasks and processes described herein may be distributed across computer systems as most advantageously suits a particular implementation, as would be understood by those skilled in the art. For example, in one embodiment, the processor programmed to perform the analysis may reside in the same system as the database, and input is received from and output is provided to the user via a thin client over a network. In another embodiment, it may be preferable to distribute the computational processes of updating disease and clinical impression as described herein to a local computer system, with the likelihood matrices residing in a database on a separate server accessible to each such system. Finally, it may be advantageous to have a standalone system, for example in a notebook or tablet computer, that contains the processor, the database, and all necessary instructions to receive data and perform the processes described herein. The computer systems may be general purpose programmable computers or they may contain hardware specially designed to perform the probabilistic methods described herein in parallel on a large scale to achieve very rapid simultaneous estimation of multiple disease conditions.

In one embodiment, the processor 84 is in communication with a computer memory having instructions to execute the prediction process herein. Prior to the clinician's performing a test or otherwise receiving a test outcome, the processor analyzes the likely effect of the available tests on a current disease impression of interest of the patient, in accordance with the prediction process. The processor then identifies to the clinician at least one test that is predicted to have diagnostic value with respect to the disease impression, and in a preferred embodiment, a plurality of tests having diagnostic value with respect to the disease impression with an indication of the predicted diagnostic progress achieved by each test. In yet another embodiment, the processor performs this process with respect to the clinical impression of the patient, that is, the collection of all disease impressions of interest for the patient, and provides an array of tests with predicted diagnostic value and the predicted diagnostic progress for each such test with respect to each disease impression for which the test has meaningful diagnostic value.

As a layer of medical logic within an electronic medical record system, the methods of the present invention support both computer-aided medical diagnosis and evidence-based medicine. A comprehensive discussion of how the methods of the present invention can address the escalating costs of healthcare is beyond the scope of this application. Briefly, the methods of the present invention allow the value of clinical information to be inferred and predicted in real time. Reduced cost may be possible when these methods operate as a supporting layer of logic within a electronic medical record system. The ability to calculate the expected change in the clinical impression from a test permits a real time cost-benefit analysis of test options. The prevalence of such analyses will only increase as we enter an era of “accountable healthcare” with medical reimbursements transitioning from pay for performance to bundled payments and other novel payment schemes.

Although the present invention has been described and shown with reference to certain preferred embodiments thereof, other embodiments are possible. The foregoing description is therefore considered in all respects to be illustrative and not restrictive. Therefore, the present invention should be defined with reference to the claims and their equivalents, and the spirit and scope of the claims should not be limited to the description of the preferred embodiments contained herein.

What is claimed:

1. A method of inferring the impression of the state of a disease in a patient, wherein said disease has a set of possible disease conditions, said method comprising:
(a) identifying a current disease impression of the patient;
(b) obtaining the outcome of a test performed with respect to said disease in said patient; and
(c) updating said current disease impression to a new disease impression based upon the conditional probabilities of obtaining said test outcome given each of said disease conditions.

2. The method of claim 1, wherein said test has a set of possible outcomes, and further comprising providing a likelihood matrix comprising the conditional probability distribution of obtaining each test outcome given each of said disease conditions, wherein the conditional probabilities in step (c) are selected from said likelihood matrix.

3. The method of claim 2, further comprising
(d) providing a plurality of tests, each of said plurality of tests having a set of possible outcomes;
(e) providing for each of said plurality of tests a likelihood matrix comprising the conditional probability distribution of each of said test outcomes given each of said disease conditions; and
(f) repeating steps (a)-(c) with respect to at least one of said plurality of tests.

4. The method of claim 3, wherein steps (a)-(c) are repeated until at least one updated disease impression is within a predetermined threshold with respect to one of said disease conditions.

5. The method of claim 3, further comprising
(g) defining for each of a plurality of diseases a set of possible disease conditions;
(h) providing a likelihood matrix for each of said plurality of tests with the conditional probability distribution of each of said test outcomes given each of said disease conditions for each of said plurality of diseases; and
(i) performing step (f) for each of said diseases, wherein each of the current disease impressions for each of said diseases is updated in parallel to a new disease impression based upon the conditional probabilities of obtaining said test outcome given each of said disease conditions selected from the corresponding likelihood matrix, such that the clinical impression of said patient is provided.

6. The method of claim 5, wherein steps (a)-(c) are repeated until at least one updated disease impression is within a predetermined threshold with respect to one of said disease conditions for at least one of said diseases.

7. The method of claim 1, wherein said test may randomly produce erroneous outcomes, whereby said random error causes the disease impression to update stochastically according to a hidden Markov model.

8. The method of claim 2, wherein said disease conditions are mutually exclusive and exhaustive.

9. The method of claim 3, wherein each of said tests is conditionally independent of one another.

10. The method of claim 3, wherein at least two of said tests are conditionally dependent upon one another.

11. The method of claim 5, wherein said at least two of said diseases may exist simultaneously in said patient.

12. The method of claim 1, wherein said current disease impression in step (a) is identified based upon the prevalence of said disease in the population demographic corresponding to said patient.

13. A method of large scale medical diagnosis comprising:
(a) storing in a database a plurality of likelihood matrices for a plurality of diseases and a plurality of tests, each said disease comprising a set of disease conditions and each said test comprising a set of possible test outcomes, each said likelihood matrix comprising for one of said diseases and one of said tests the conditional probability distribution of each test outcome given each disease condition;
(b) identifying a current disease impression for a plurality of diseases of a patient;
(c) obtaining the outcome of a test performed in said patient;
(d) retrieving from said database the conditional probabilities of obtaining said test outcome given each of said disease conditions of each of said diseases; and
(e) updating in parallel each of said current disease impressions to a new disease impression based upon said conditional probabilities, said plurality of new disease impressions forming the clinical impression of said patient.

14. The method of claim 13, wherein steps (b)-(e) are repeated until at least one new disease impression is within a predetermined threshold with respect to one of said disease conditions for at least one of said diseases.

15. The method of claim 13, wherein said test may randomly produce erroneous outcomes, whereby said random error causes the disease impression to update stochastically according to a hidden Markov model.

16. The method of claim 13, wherein said disease conditions are mutually exclusive and exhaustive.

17. The method of claim 13, wherein each of said tests is conditionally independent of one another.

18. The method of claim 13, wherein at least two of said tests are conditionally dependent upon one another.

19. The method of claim 13, wherein said at least two of said diseases may exist simultaneously in said patient.

20. The method of claim 13, wherein said current disease impression in step (a) is identified based upon the prevalence of said disease in the population demographic corresponding to said patient.

21. A system comprising
a database stored in a computer readable memory comprising a plurality of likelihood matrices for a plurality of diseases and a plurality of tests, each said disease comprising a set of disease conditions and each said test comprising a set of possible test outcomes, each said likelihood matrix comprising for one of said diseases and one of said tests the conditional probability distribution of each test outcome given each disease condition;
a processor in communication with said database, said processor having a computer readable memory storing instructions executable by said processor, said instructions comprising:
(a) identifying a current disease impression of a patient;
(b) obtaining the outcome of a test performed with respect to said disease in said patient;
(c) retrieving from said database the conditional probabilities of obtaining said test outcome given each of said disease conditions; and
(d) updating said current disease impression to a new disease impression based upon said conditional probabilities.

22. The system of claim 21, wherein in step (a) said processor receives said current disease impression from a user.

23. The system of claim 21, wherein in step (a) said processor obtains said current disease impression from a medical record of said patient.

24. The system of claim 21, wherein, prior to step (b), communicating said current disease impression to said database and receiving from said database the identity of at least one test predicted to have diagnostic value based upon said current disease impression and said likelihood matrices, and wherein the test performed in step (b) is selected from the at least one test so identified.

25. The system of claim 21, further comprising
(e) comparing the new disease impression to a predetermined threshold for diagnosis of one of said disease conditions for at least one of said diseases, and if said new disease impression is within said threshold, communicating said diagnosis to a user.

26. The system of claim 25, further comprising, updating a medical record with said diagnosis.

27. A system comprising
a database stored in a computer readable memory comprising a plurality of likelihood matrices for a plurality of diseases and a plurality of tests, each said disease comprising a set of disease conditions and each said test comprising a set of possible test outcomes, each said likelihood matrix comprising for one of said diseases and one of said tests the conditional probability distribution of each test outcome given each disease condition;
a processor in communication with said database, said processor having a computer readable memory storing instructions executable by said processor, said instructions comprising:
(a) identifying a current disease impression for each of a plurality of diseases of a patient;
(b) obtaining the outcome of a test performed in said patient;
(c) retrieving from said database the conditional probabilities of obtaining said test outcome given each disease condition for each of said plurality of diseases; and
(d) updating in parallel each of said current disease impressions to a new disease impression based upon said conditional probabilities, said plurality of new disease impressions forming the clinical impression of said patient.
